(19) United States
(12) Patent Application Publication
(10) Pub. No.: US 2017/0172891 A1
(43) Pub. Date: Jun. 22, 2017

(54) PRODUCTS AND METHOD FOR THE TEMPORARY SHAPING OF KERATIN-CONTAINING FIBERS

(71) Applicant: Henkel AG & Co. KGaA, Duesseldorf (DE)

(72) Inventors: Julia Bibiane Lange, Bad Bramstedt (DE); Bernd Richters, Hamburg (DE); Maria Catalina Bermudez Agudelo, Duesseldorf (DE); Cyrielle Martinez, Hamburg (DE)

(73) Assignee: Henkel AG & Co. KGaA, Duesseldorf (DE)

(21) Appl. No.: 15/448,861

(22) Filed: Mar. 3, 2017

Related U.S. Application Data
(63) Continuation of application No. PCT/EP2015/064184, filed on Jun. 24, 2015.

(30) Foreign Application Priority Data
Sep. 3, 2014 (DE) 10 2014 217 602.6

(51) Int. Cl. A61K 8/81; A61K 8/91; A61Q 5/06

(52) U.S. Cl. CPC A61K 8/8152; A61Q 5/06; A61K 8/91; A61K 8/8176; A61K 8/8182; A61K 8/8135; A61K 2800/594; A61K 2800/54

(57) ABSTRACT

The invention relates to cosmetic products for temporarily shaping keratin fibers, including, relative to the total weight of the product, a) 0.5 to 3.0 wt.-% of at least one hydrophobically modified (meth)acrylic acid copolymer; and b) 0.5 to 5.0 wt.-% of at least one copolymer which is different from copolymer a) and which is obtained by reacting at least one ethylenically unsaturated monomer unit (b1) with at least one saccharide monomer unit (b2), and to the use thereof and to a method for using said products.

### FIELD OF THE INVENTION

The present invention relates to a cosmetic product for setting hair and/or for temporarily shaping keratin fibers, in particular human hair, the product including a combination of two polymers.

### BACKGROUND OF THE INVENTION

The temporary creation of hairstyles for a relatively long duration of up to several days generally requires the use of active setting substances. Hair treatment agents which serve to temporarily shape the hair therefore play an important role. Suitable temporary shaping products usually include synthetic polymers and/or waxes as the active setting substance. Products for helping to temporarily shape keratin-containing fibers may for example be in the form of hairspray, hair wax, hair gel or hair mousse.

The most important property of a product for temporarily shaping hair, hereinafter also referred to as a styling product, consists in giving the strongest possible hold to the treated fibers in the newly configured shape, that is to say a shape that is impressed on the hair. This is also referred to as a strong hairstyle hold or a high degree of hold of the styling product. The hairstyle hold is determined substantially by the type and amount of the active setting substances used, but the other constituents of the styling product may also have an influence.

Besides a high degree of hold, styling products must also satisfy a number of further requirements. These can be subdivided roughly into properties on the hair, properties of the respective formulation, for example properties of the mousse, gel or sprayed aerosol, and properties relating to the handling of the styling product, particular importance being assigned to the properties on the hair. Mention may be made in particular of moisture resistance, strength, low tack and a balanced conditioning effect. Furthermore, a styling product should be able to be used universally where possible for all hair types and should be mild on the hair and skin.

To satisfy the various requirements, a large number of synthetic polymers have already been developed as active setting substances, and these are used in styling products. The polymers can be subdivided into cationic, anionic, nonionic and amphoteric setting polymers. Ideally, when applied to the hair, the polymers create a polymer film which on the one hand gives the hairstyle strong hold but on the other hand is sufficiently flexible not to break under stress.

One example of a styling product with good hold properties and high moisture resistance, which is based on a combination of a hydrophobically modified (meth)acrylic acid copolymer and a hydrophobically modified polysaccharide, is described in the international patent application WO 2014/26804 A2.

Copolymers suitable for temporarily shaping hair, which are obtained by reacting ethylenically unsaturated monomer units with saccharide monomer units, for example by reacting vinylpyrrolidone with maltodextrin, are described in the international application WO 2011/017223 A1.

Furthermore, other desirable features and characteristics of the present invention will become apparent from the subsequent detailed description of the invention and the appended claims, taken in conjunction with this background of the invention.

### BRIEF SUMMARY OF THE INVENTION

This object was able to be achieved by combining two specific polymers. A first subject matter of the present invention is therefore a cosmetic product for temporarily shaping keratin fibers, including, relative to the total weight of the product,
a) 0.5 to 3.0 wt.-% of at least one hydrophobically modified (meth)acrylic acid copolymer;
b) 0.5 to 5.0 wt.-% of at least one copolymer which is different from the copolymer a) and which is obtained by reacting at least one ethylenically unsaturated monomer unit (b 1) with at least one saccharide monomer unit (b2).

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

One object of the present invention was to provide further suitable polymer combinations which are characterized by good film-forming and/or setting properties, have a very high degree of hold without compromising on flexibility and good moisture resistance—in particular resistance to sweat and water—and are also suitable for producing stable viscous and preferably stable transparent cosmetic compositions. In particular, styling products available at present are still capable of improvement since a good combination of stiffness and long-term hold is not always sufficiently ensured. It is therefore an object of the present invention to provide such styling products which, besides having the abovementioned properties, provide in particular both a good stiffness and also a good long-term hold.

A first essential constituent of cosmetic products according to the invention is the hydrophobically modified (meth)acrylic acid copolymer a). Preferred copolymers a) have a thickening effect.

As hydrophobically modified (meth)acrylic acid copolymers a), use is preferably made of copolymers which can be attributed to
at least one monomer (al) from the group consisting of unsaturated carboxylic acids and unsaturated carboxylic acid esters, and
at least one monomer (a2) from the group consisting of unsaturated hydrophobically modified monomers.

Preferred copolymers a) are based on at least one monomer (a1) from the group consisting of acrylic acid, methacrylic acid, C1-C6 alkyl acrylic acid esters, C1-C6 alkyl methacrylic acid esters. The acrylic acid esters and methacrylic acid esters are preferably esters of the respective acids with non-tertiary alkyl alcohols having alkyl radicals of 1 to 12 carbon atoms, in particular 2 to 4 carbon atoms. As suitable monomers, mention may be made for example of ethyl acrylate, ethyl methacrylate, propyl acrylate, propyl methacrylate, n-butyl acrylate, n-butyl methacrylate, isobutyl acrylate, 2-methylbutyl acrylate, 2-ethylhexyl acrylate, n-octyl acrylate, isooctyl acrylate, isooctyl methacrylate, isononyl acrylate and isodecyl acrylate.

The group of hydrophobically modified monomers (a2) refers to monomers which have a hydrophobic partial structure. Preferred monomers (a2) can in turn be attributed to the following two structural units:
an unsaturated acid, preferably acrylic acid, methacrylic acid or itaconic acid;
a C8-40 alkyl chain, preferably a C10-30 alkyl chain.

These two partial structures may optionally be supplemented with a third structural unit from the group consisting of polyoxyalkylene groups, preferably polyethylene glycol groups, polypropylene glycol groups or polyethylene glycol/polypropylene glycol groups.

As the monomer (a2), use is made for example of C10-30 alkyl acrylates, C10-30 alkyl PEG acrylates, C10-30 alkyl PEG methacrylates or C10-30 alkyl PEG itaconates. Preferred monomers (a2) are selected from C10-30 alkyl acrylates, C10-30 alkyl PEG 20-25 acrylates, C10-30 alkyl PEG 20-25 methacrylates or C10-30 alkyl PEG 20-25 itaconates. Particularly preferred monomers (a2) are selected from the group consisting of C10-30 alkyl acrylates, steareth-20 methacrylate, beheneth-25 methacrylate, steareth-20 itaconate, ceteth-20 itaconate, palmeth-25 acrylate or C10-30 alkyl PEG 20 itaconates.

To sum up, preference is given to copolymers a) which are formed from
at least one monomer (a1) from the group consisting of acrylic acid, methacrylic acid, C1-C6 alkyl acrylic acid esters, C1-C6 alkyl methacrylic acid esters,
at least one monomer (a2) from the group consisting of C10-30 alkyl acrylates,
C10-30 alkyl PEG acrylates, C10-30 alkyl PEG methacrylates or C10-30 alkyl PEG itaconates.

Particular preference is given to copolymers a) which are obtained by reacting
at least one monomer (a1) from the group consisting of acrylic acid, methacrylic acid, C1-C6 alkyl acrylic acid esters, C1-C6 alkyl methacrylic acid esters with
at least one monomer (a2) from the group consisting of C10-30 alkyl acrylates, C10-30 alkyl methacrylates, C10-30 alkyl PEG acrylates, C10-30 alkyl PEG methacrylates or C10-30 alkyl PEG itaconates.

Other preferred hydrophobically modified (meth)acrylic acid copolymers a) are formed not only from the previously described monomers (a1) and (a2) but also from at least one monomer (a3) from the group of unsaturated amine-group-containing monomers.

As the monomer (a3), use is preferably made of monomers from the group consisting of acrylamide, methacrylamide, mono-(C1-C4)-alkylamino-(C1-C4)-alkyl acrylate, di-(C1-C4)- alkylamino-(C1-C4)- alkyl acrylate, mono-(C1-C4)-alkylamino-(C1-C4)-alkyl methacrylate, di-(C1-C4)-alkylamino-(C1-C4)-alkyl methacrylate.

Exemplary and preferred monomers (A2) are 2-(N,N-dimethylamino)ethyl acrylate, 2-(N,N-dimethylamino)ethyl methacrylate, 2-(N,N-diethylamino)ethyl acrylate, 2-(N,N-diethylamino)ethyl methacrylate, 3-(N,N-dimethylamino)propyl acrylate, 3-(N,N-dimethylamino)propyl methacrylate, 2-(N,N-dimethylamino)neopentyl acrylate, N′-(3-N,N-dimethylamino)propyl acrylamide, N′ -(3 -N,N-dimethylamino)propyl methacrylamide.

Preference is given to copolymers A formed from
at least one monomer (a1) from the group consisting of acrylic acid, methacrylic acid, C1-C6 alkyl acrylic acid esters, C1-C6 alkyl methacrylic acid esters,
at least one monomer (a2) from the group consisting of C10-30 alkyl PEG 20 itaconates,
at least one monomer (a3) from the group consisting of acrylamide, methacrylamide, mono-(C1-C4)-alkylamino-(C1-C4)-alkyl acrylate, di-(C1-C4)-alkylamino-(C1-C4)-alkyl acrylate, mono-(C1-C4)-alkylamino-(C1-C4)-alkyl methacrylate, di-(C1-C4)-alkylamino-(C1-C4)-alkyl methacrylate.

Copolymers a) which are preferably used are not crosslinked.

To sum up, cosmetic products which are preferred according to the invention are characterized in that the copolymer a) is selected from the group of compounds having the INCI names Acrylates/C10 30 Alkyl Methacrylate Copolymer, Acrylates/C10-30 Alkyl Acrylate Crosspolymer, Acrylates/Steareth-20 Methacrylate Crosspolymer, Acrylates/Steareth-20 Methacrylate Copolymer, Acrylates/Beheneth-25 Methacrylate Copolymer, Acrylates/Steareth-20 Itaconate Copolymer, Acrylates/Ceteth-20 Itaconate Copolymer, Acrylates/Palmeth-25 Acrylate Copolymer, Acrylates/Aminoacrylates/C10-30 Alkyl PEG-20 Itaconate Copolymer. Suitable polymers are available for example under the trade names Luvigel® FIT, Ultrez® 21, Pemulen® TR1, Aculyn® 22, Aculyn® 28, Aculyn® 88, Structure® 2001, Structure® 3001, Synthalen® W2000 and Structure® Plus. With particular preference, the copolymer a) is selected from the group of compounds having the INCI name Acrylates/C10-30 Alkyl Methacrylate Copolymer.

For the cosmetic effect of products according to the invention, it has proven to be advantageous to further limit the proportion by weight of the copolymer a) in the total weight of the cosmetic product. Preferred cosmetic products are therefore characterized in that the proportion by weight of the copolymer a) in the total weight of the cosmetic product is 0.7 to 2.0 wt.-%, preferably 0.9 to 1.2 wt. %.

As a second essential constituent, the cosmetic products according to the invention include at least one copolymer b) which is different from the copolymer a) and which is obtained by reacting at least one ethylenically unsaturated monomer unit (b1) with at least one saccharide monomer unit (b2). Preferred copolymers b) have no thickening effect. The proportion by weight of the copolymer b) in the total weight of the cosmetic product is preferably 1.0 to 4.0 wt.-%, particularly preferably 1.5 to 3.0 wt.-%.

The copolymers b) are preferably prepared by reacting a growing synthetic polymer chain of the unsaturated monomer unit (b1) with a saccharide monomer unit (b2). It is assumed that the reaction takes place via a mechanism in which
by means of an initiator, free radicals are formed which react with the unsaturated monomer unit (b1) and initiate the formation of a synthetic polymer chain composed of monomer units (b1),
the chain transfer to form a (b1) homopolymer is ended inter alia by abstracting a proton from the saccharide monomer unit (b2),
the resulting radical of the saccharide monomer unit (b2) reacts with an unsaturated monomer unit (b1) and in this way initiates the formation of the copolymer b).

A fully reacted reaction mixture of the monomer units (b1) and (b2) will usually include, besides the copolymer b), also inter alia (b1) homopolymers and residues of the saccharide monomer b2).

The saccharide monomer unit (b2) will usually form the end group of the copolymer b) as a consequence of the reaction mechanism described above.

The copolymer b) is preferably soluble in water and water/ethanol mixtures. The solubility in water (25° C.) is preferably more than 1 g/l, more preferably more than 10 g/I.

As ethylenically unsaturated monomer units (b1), use may be made of anionic, cationic or nonionic monomers, preference being given to the use of nonionic monomer units. The group of preferred nonionic monomers (b1) includes acrylamide, methacrylamide, N-alkyl (meth)acrylamide, N,N-dialkyl (meth)acrylamide, such as N,N-dimethyl acrylamide, hydroxyalkyl (meth)acrylates, alkyl (meth)acrylates, in particular methyl acrylate and methyl methacrylate, vinyl acetate, vinylmorpholine, vinylpyrrolidone, vinylcaprolactam, vinylformamide, vinylacetamide, ethoxylated alkyl, alkaryl or aryl monomers such as methoxypolyethylene glycol (meth)acrylate, allyl glycidyl ether, allyl alcohol, glycerol (meth)acrylate monomers silane, silanol and siloxane functionalities and others. The nonionic ethylenically unsaturated monomer (b2) is preferably water-soluble.

The preferred nonionic ethylenically unsaturated monomers (b1) are acrylamide, methacryl amide, N-methyl (meth)acrylamide, N,N-di methyl (meth)acrylamide, vinylpyrrolidone, vinylformamide, vinylacetamide and vinylcaprolactam. Vinylpyrrolidone is very particularly preferred.

As saccharide monomer units (b2), use may be made of monosaccharides, disaccharides but also oligosaccharides and polysaccharides. Suitable polysaccharides are for example chitin, chitosan, gum arabic, agar, algin, alginates, carrageenan, xanthan, gellan, welan, rhamsan, curdlan, scleroglucan, tamarind gum, hemicellulose, cellulose, carboxymethyl cellulose, hydroxyethyl cellulose, hydroxypropyl cellulose, ethyl methyl hydroxyethyl cellulose d-xylane, maltodextrin, corn syrup, starch, oxidized starch, chemically modified starches, inulin or inulin carboxymethyl cellulose. Particular preference is given to the use of starch and starch derivatives, for example the use of the maltodextrin obtained by hydrolysis of starch or of the pyrodextrin obtainable by heating starch.

The maltodextrin obtainable by hydrolysis of starch is a water-soluble carbohydrate mixture of monomers, dimers, oligomers and polymers of glucose. The use of maltodextrin as the monomer (b2) is particularly preferred.

To sum up, preferred copolymers b) are thus obtained by reacting
at least one monomer (b 1) from the group consisting of N-vinylpyrrolidone and N-vinylformamide, preferably N-vinylpyrrolidone, with
at least one monomer (b2) from the group consisting of modified starches, preferably the hydrolyzed starches, particularly preferably from the group of maltodextrins.

One particularly preferred embodiment of cosmetic products according to the invention is characterized in that the copolymer b) is selected from the group of compounds having the INCI name Maltodextrin/VP Copolymer.

Besides the copolymers a) and copolymers b) described above, the cosmetic products according to the invention may include further active substances, auxiliaries and care substances.

A first group of active substances used with preference is formed by the film-forming polymers. These film-forming polymers are not identical to the hydrophobically modified (meth)acrylic acid copolymer a) described above. The proportion by weight of the film-forming polymer in the total weight of the cosmetic product is preferably 0.1 to 8.0 wt.-%, more preferably 0.5 to 6.0 wt.-% and in particular 1.0 to 4.0 wt.-%.

With particular preference, nonionic polymers are used as the film-forming polymers. Suitable nonionic polymers are for example:
vinylpyrrolidone/vinyl ester copolymers, as sold for example under the trademark Luviskol® (BASF). Luviskol® VA 64 and Luviskol® VA 73, each vinylpyrrolidone/vinyl acetate copolymers, are preferred nonionic polymers.
cellulose ethers, such as hydroxypropyl cellulose, hydroxyethyl cellulose and methyl hydroxypropyl cellulose, as sold for example under the trademarks Culminal® and Benecel® (AQUALON).
shellac.
polyvinylpyrrolidones, as sold for example under the name Luviskol® (BASF).
siloxanes. These siloxanes may be either water-soluble or water-insoluble. Both volatile and nonvolatile siloxanes are suitable, wherein nonvolatile siloxanes are understood to mean those compounds having a boiling point above 200° C. at atmospheric pressure. Preferred siloxanes are polydialkylsiloxanes, such as for example polydimethylsiloxane, polyalkylarylsiloxanes, such as for example polyphenylmethylsiloxane, ethoxylated polydialkylsiloxanes, and polydialkylsiloxanes which include amine and/or hydroxyl groups.
glycoside-substituted silicones.

Due to their cosmetic effect in combination with the copolymers a) and b), film-forming polymers which are preferably used according to the invention are in particular the polyvinylpyrrolidones (INCI name: PVP) and the vinylpyrrolidone/vinyl acetate copolymers (INCI name: VP/VA Copolymer), wherein the proportion by weight of these polymers is preferably limited to amounts between 1.0 and 10 wt.-%. Particularly preferred cosmetic products according to the invention are therefore characterized in that they also include, relative to the total weight of the product, 1.0 to 10 wt.-% polyvinylpyrrolidone and/or vinylpyrrolidone/vinyl acetate copolymer, preferably polyvinylpyrrolidone. Particularly preferred cosmetic products have a proportion by weight of the polyvinylpyrrolidone and/or vinylpyrrolidone/vinyl acetate copolymer c) in the total weight of the cosmetic product of from 2.0 to 8.5 wt.-%, preferably 3.0 to 7.0 wt.-%.

To sum up, cosmetic products which are particularly preferred according to the invention include, with the copolymers a) and b) and the film-forming polymer c), three polymers which differ from one another. In one preferred embodiment, the cosmetic products include, relative to the total weight of the product,
a) 0.5 to 3.0 wt.-% of a copolymer a), which is obtained by reacting
at least one monomer (A1) from the group consisting of acrylic acid, methacrylic acid, C1-C6 alkyl acrylic acid esters, C1-C6 alkyl methacrylic acid esters with
at least one monomer (A2) from the group consisting of C10-30 alkyl acrylates, C10-30 alkyl methacrylates, C10-30 alkyl PEG acrylates, C10-30 alkyl PEG methacrylates or C10-30 alkyl PEG itaconates;
b) 0.5 to 5.0 wt.-% of a copolymer b), which is obtained by reacting N-vinylpyrrolidone (B1) with at least one monomer (B2) from the group of maltodextrins;
c) to 10 wt.-% polyvinylpyrrolidone.

Particular preference is given to cosmetic products which include, relative to the total weight of the product,
a) 0.9 to 1.2 wt.-% of a copolymer a) from the group of compounds having the INCI name Acrylates/C10-30 Alkyl Methacrylate Copolymer;
b) 1.5 to 3.0 wt.-% of a copolymer b) from the group of compounds having the INCI name Maltodextrin/VP Copolymer;
c) 3.0 to 7.0 wt.-% polyvinylpyrrolidone.

As a care substance, use may be made of protein hydrolysates and/or derivatives thereof. Protein hydrolysates are product mixtures obtained by acid-, base- or enzyme-catalyzed degradation of proteins (albumins). According to the invention, the term protein hydrolysates is also understood to mean total hydrolysates and also individual amino acids and derivatives thereof as well as mixtures of different amino acids. The molecular weight of the protein hydrolysates which can be used according to the invention is between 75, the molecular weight of glycine, and 200,000, and preferably the molecular weight is 75 to 50,000 and very particularly preferably 75 to 20,000 Daltons.

A further group of care substances is formed by vitamins, provitamins, vitamin precursors and/or derivatives thereof. According to the invention, preference is given to those vitamins, provitamins and vitamin precursors which are usually assigned to groups A, B, C, E, F and H.

Further care substances are glycerol, propylene glycol, panthenol, caffeine, nicotinamide and sorbitol.

As a care substance, use may be made of plant extracts, but also mono- or oligosaccharides and/or lipids.

Preferred cosmetic products are based on an aqueous or aqueous/alcoholic carrier. The proportion of water in the total weight of cosmetic products according to the invention is preferably 80 to 99 wt. %, more preferably 84 to 98 wt.-% and in particular 90 to 97 wt.-%.

The composition of some cosmetic products which are used can be found in the following tables (unless otherwise indicated, amounts are given in wt.-% relative to the total weight of the cosmetic product).

| | Formula | Formula | Formula | Formula | Formula |
|---|---|---|---|---|---|
| | 1 | 2 | 3 | 4 | 5 |
| Copolymer a) ¹⁾ | 0.5 to 3.0 | 0.5 to 3.0 | 0.7 to 2.0 | 0.7 to 2.0 | 0.9 to 1.2 |
| Copolymer b) ²⁾ | 0.5 to 5.0 | 1.0 to 4.0 | 1.0 to 4.0 | 1.5 to 3.0 | 1.5 to 3.0 |
| Optional | ad 100 | ad 100 | ad 100 | ad 100 | ad 100 |
| additives | | | | | |
| | Formula | Formula | Formula | Formula | Formula |
| | 6 | 7 | 8 | 9 | 10 |
| Copolymer a) ¹⁾ | 0.5 to 3.0 | 0.5 to 3.0 | 0.7 to 2.0 | 0.7 to 2.0 | 0.9 to 1.2 |
| Copolymer b) ²⁾ | 0.5 to 5.0 | 1.0 to 4.0 | 1.0 to 4.0 | 1.5 to 3.0 | 1.5 to 3.0 |
| Water | 80 to 99 | 82 to 99 | 84 to 98 | 87 to 98 | 90 to 97 |
| Optional | add 100 | add 100 | add 100 | add 100 | add 100 |
| additives | | | | | |
| | Formula | Formula | Formula | Formula | Formula |
| | 11 | 12 | 13 | 14 | 15 |
| Copolymer a) ³⁾ | 0.5 to 3.0 | 0.5 to 3.0 | 0.7 to 2.0 | 0.7 to 2.0 | 0.9 to 1.2 |
| Copolymer b) ²⁾ | 0.5 to 5.0 | 1.0 to 4.0 | 1.0 to 4.0 | 1.5 to 3.0 | 1.5 to 3.0 |
| Optional | ad 100 | ad 100 | ad 100 | ad 100 | ad 100 |
| additives | | | | | |
| | Formula | Formula | Formula | Formula | Formula |
| | 16 | 17 | 18 | 19 | 20 |
| Copolymer a) ³⁾ | 0.5 to 3.0 | 0.5 to 3.0 | 0.7 to 2.0 | 0.7 to 2.0 | 0.9 to 1.2 |
| Copolymer b) ²⁾ | 0.5 to 5.0 | 1.0 to 4.0 | 1.0 to 4.0 | 1.5 to 3.0 | 1.5 to 3.0 |
| Water | 80 to 99 | 82 to 99 | 84 to 98 | 87 to 98 | 90 to 97 |
| Optional | add 100 | add 100 | add 100 | add 100 | add 100 |
| additives | | | | | |
| | Formula | Formula | Formula | Formula | Formula |
| | 21 | 22 | 23 | 24 | 25 |
| Copolymer a) ¹⁾ | 0.5 to 3.0 | 0.5 to 3.0 | 0.7 to 2.0 | 0.7 to 2.0 | 0.9 to 1.2 |
| Copolymer b) ⁴⁾ | 0.5 to 5.0 | 1.0 to 4.0 | 1.0 to 4.0 | 1.5 to 3.0 | 1.5 to 3.0 |
| Optional | ad 100 | ad 100 | ad 100 | ad 100 | ad 100 |
| additives | | | | | |
| | Formula | Formula | Formula | Formula | Formula |
| | 26 | 27 | 28 | 29 | 30 |
| Copolymer a) ¹⁾ | 0.5 to 3.0 | 0.5 to 3.0 | 0.7 to 2.0 | 0.7 to 2.0 | 0.9 to 1.2 |
| Copolymer b) ⁴⁾ | 0.5 to 5.0 | 1.0 to 4.0 | 1.0 to 4.0 | 1.5 to 3.0 | 1.5 to 3.0 |
| Water | 80 to 99 | 82 to 99 | 84 to 98 | 87 to 98 | 90 to 97 |
| Optional | ad 100 | ad 100 | ad 100 | ad 100 | ad 100 |
| additives | | | | | |
| | Formula | Formula | Formula | Formula | Formula |
| | 31 | 32 | 33 | 34 | 35 |
| Copolymer a) ³⁾ | 0.5 to 3.0 | 0.5 to 3.0 | 0.7 to 2.0 | 0.7 to 2.0 | 0.9 to 1.2 |
| Copolymer b) ⁴⁾ | 0.5 to 5.0 | 1.0 to 4.0 | 1.0 to 4.0 | 1.5 to 3.0 | 1.5 to 3.0 |
| Optional | ad 100 | ad 100 | ad 100 | ad 100 | ad 100 |
| additives | | | | | |
| | Formula | Formula | Formula | Formula | Formula |
| | 36 | 37 | 38 | 39 | 40 |
| Copolymer a) ³⁾ | 0.5 to 3.0 | 0.5 to 3.0 | 0.7 to 2.0 | 0.7 to 2.0 | 0.9 to 1.2 |
| Copolymer b) ⁴⁾ | 0.5 to 5.0 | 1.0 to 4.0 | 1.0 to 4.0 | 1.5 to 3.0 | 1.5 to 3.0 |
| Water | 80 to 99 | 82 to 99 | 84 to 98 | 87 to 98 | 90 to 97 |
| Optional | ad 100 | ad 100 | ad 100 | ad 100 | ad 100 |
| additives | | | | | |
| | Formula | Formula | Formula | Formula | Formula |
| | 41 | 42 | 43 | 44 | 45 |
| Acrylates/C₁₀₋₃₀ | 0.5 to 3.0 | 0.5 to 3.0 | 0.7 to 2.0 | 0.7 to 2.0 | 0.9 to 1.2 |
| Alkyl | | | | | |
| Methacrylate | | | | | |
| Copolymer | | | | | |
| Maltodextrin/ | 0.5 to 5.0 | 1.0 to 4.0 | 1.0 to 4.0 | 1.5 to 3.0 | 1.5 to 3.0 |
| VP Copolymer | | | | | |
| Optional | ad 100 | ad 100 | ad 100 | ad 100 | ad 100 |
| additives | | | | | |
| | Formula | Formula | Formula | Formula | Formula |
| | 46 | 47 | 48 | 49 | 50 |
| Acrylates/C₁₀₋₃₀ | 0.5 to 3.0 | 0.5 to 3.0 | 0.7 to 2.0 | 0.7 to 2.0 | 0.9 to 1.2 |
| Alkyl | | | | | |
| Methacrylate | | | | | |
| Copolymer | | | | | |
| Maltodextrin/ | 0.5 to 5.0 | 1.0 to 4.0 | 1.0 to 4.0 | 1.5 to 3.0 | 1.5 to 3.0 |
| VP Copolymer | | | | | |
| Water | 80 to 99 | 82 to 99 | 84 to 98 | 87 to 98 | 90 to 97 |
| Optional | ad 100 | ad 100 | ad 100 | ad 100 | ad 100 |
| additives | | | | | |
| | Formula | Formula | Formula | Formula | Formula |
| | 51 | 52 | 53 | 54 | 55 |
| Copolymer a) ¹⁾ | 0.5 to 3.0 | 0.5 to 3.0 | 0.7 to 2.0 | 0.7 to 2.0 | 0.9 to 1.2 |
| Copolymer b) ²⁾ | 0.5 to 5.0 | 1.0 to 4.0 | 1.0 to 4.0 | 1.5 to 3.0 | 1.5 to 3.0 |
| Polyvinyl- | 1.0 to 10 | 2.0 to 9.0 | 2.5 to 8.5 | 2.0 to 8.0 | 3.0 to 7.0 |
| pyrrolidone | | | | | |
| and/or | | | | | |
| vinyl- | | | | | |
| pyrrolidone/ | | | | | |
| vinyl | | | | | |
| acetate | | | | | |
| copolymer | | | | | |
| Optional | ad 100 | ad 100 | ad 100 | ad 100 | ad 100 |
| additives | | | | | |
| | Formula | Formula | Formula | Formula | Formula |
| | 56 | 57 | 58 | 59 | 60 |
| Copolymer a) ¹⁾ | 0.5 to 3.0 | 0.5 to 3.0 | 0.7 to 2.0 | 0.7 to 2.0 | 0.9 to 1.2 |
| Copolymer b) ²⁾ | 0.5 to 5.0 | 1.0 to 4.0 | 1.0 to 4.0 | 1.5 to 3.0 | 1.5 to 3.0 |
| Polyvinyl- | 1.0 to 10 | 2.0 to 9.0 | 2.5 to 8.5 | 2.0 to 8.0 | 3.0 to 7.0 |
| pyrrolidone | | | | | |
| and/or | | | | | |
| vinyl- | | | | | |
| pyrrolidone/ | | | | | |
| vinyl | | | | | |
| acetate | | | | | |
| copolymer | | | | | |
| Water | 80 to 99 | 82 to 99 | 84 to 98 | 87 to 98 | 90 to 97 |
| Optional | ad 100 | ad 100 | ad 100 | ad 100 | ad 100 |
| additives | | | | | |
| | Formula | Formula | Formula | Formula | Formula |
| | 61 | 62 | 63 | 64 | 65 |
| Copolymer a) ³⁾ | 0.5 to 3.0 | 0.5 to 3.0 | 0.7 to 2.0 | 0.7 to 2.0 | 0.9 to 1.2 |
| Copolymer b) ²⁾ | 0.5 to 5.0 | 1.0 to 4.0 | 1.0 to 4.0 | 1.5 to 3.0 | 1.5 to 3.0 |
| Polyvinyl- | 1.0 to 10 | 2.0 to 9.0 | 2.5 to 8.5 | 2.0 to 8.0 | 3.0 to 7.0 |
| pyrrolidone | | | | | |
| and/or | | | | | |
| vinyl- | | | | | |
| pyrrolidone/ | | | | | |
| vinyl | | | | | |
| acetate | | | | | |
| copolymer | | | | | |
| Optional | ad 100 | ad 100 | ad 100 | ad 100 | ad 100 |
| additives | | | | | |
| | Formula | Formula | Formula | Formula | Formula |
| | 66 | 67 | 68 | 69 | 70 |
| Copolymer a) ³⁾ | 0.5 to 3.0 | 0.5 to 3.0 | 0.7 to 2.0 | 0.7 to 2.0 | 0.9 to 1.2 |
| Copolymer b) ²⁾ | 0.5 to 5.0 | 1.0 to 4.0 | 1.0 to 4.0 | 1.5 to 3.0 | 1.5 to 3.0 |
| Polyvinyl- | 1.0 to 10 | 2.0 to 9.0 | 2.5 to 8.5 | 2.0 to 8.0 | 3.0 to 7.0 |
| pyrrolidone | | | | | |
| and/or | | | | | |
| vinyl- | | | | | |
| pyrrolidone/ | | | | | |
| vinyl | | | | | |
| acetate | | | | | |
| copolymer | | | | | |
| Water | 80 to 99 | 82 to 99 | 84 to 98 | 87 to 98 | 90 to 97 |
| Optional | ad 100 | ad 100 | ad 100 | ad 100 | ad 100 |
| additives | | | | | |
| | Formula | Formula | Formula | Formula | Formula |
| | 71 | 72 | 73 | 74 | 75 |
| Copolymer a) ¹⁾ | 0.5 to 3.0 | 0.5 to 3.0 | 0.7 to 2.0 | 0.7 to 2.0 | 0.9 to 1.2 |
| Copolymer b) ⁴⁾ | 0.5 to 5.0 | 1.0 to 4.0 | 1.0 to 4.0 | 1.5 to 3.0 | 1.5 to 3.0 |
| Polyvinyl- | 1.0 to 10 | 2.0 to 9.0 | 2.5 to 8.5 | 2.0 to 8.0 | 3.0 to 7.0 |
| pyrrolidone | | | | | |
| and/or | | | | | |
| vinyl- | | | | | |
| pyrrolidone/ | | | | | |
| vinyl | | | | | |
| acetate | | | | | |
| copolymer | | | | | |
| Optional | ad 100 | ad 100 | ad 100 | ad 100 | ad 100 |
| additives | | | | | |
| | Formula | Formula | Formula | Formula | Formula |
| | 76 | 77 | 78 | 79 | 80 |
| Copolymer a) ¹⁾ | 0.5 to 3.0 | 0.5 to 3.0 | 0.7 to 2.0 | 0.7 to 2.0 | 0.9 to 1.2 |
| Copolymer b) ⁴⁾ | 0.5 to 5.0 | 1.0 to 4.0 | 1.0 to 4.0 | 1.5 to 3.0 | 1.5 to 3.0 |
| Polyvinyl- | 1.0 to 10 | 2.0 to 9.0 | 2.5 to 8.5 | 2.0 to 8.0 | 3.0 to 7.0 |
| pyrrolidone | | | | | |
| and/or | | | | | |
| vinyl- | | | | | |
| pyrrolidone/ | | | | | |
| vinyl | | | | | |
| acetate | | | | | |
| copolymer | | | | | |
| Water | 80 to 99 | 82 to 99 | 84 to 98 | 87 to 98 | 90 to 97 |
| Optional | ad 100 | ad 100 | ad 100 | ad 100 | ad 100 |
| additives | | | | | |
| | Formula | Formula | Formula | Formula | Formula |
| | 81 | 82 | 83 | 84 | 85 |
| Copolymer a) ³⁾ | 0.5 to 3.0 | 0.5 to 3.0 | 0.7 to 2.0 | 0.7 to 2.0 | 0.9 to 1.2 |
| Copolymer b) ⁴⁾ | 0.5 to 5.0 | 1.0 to 4.0 | 1.0 to 4.0 | 1.5 to 3.0 | 1.5 to 3.0 |
| Polyvinyl- | 1.0 to 10 | 2.0 to 9.0 | 2.5 to 8.5 | 2.0 to 8.0 | 3.0 to 7.0 |
| pyrrolidone | | | | | |
| and/or | | | | | |
| vinyl- | | | | | |
| pyrrolidone/ | | | | | |
| vinyl | | | | | |
| acetate | | | | | |
| copolymer | | | | | |
| Optional | ad 100 | ad 100 | ad 100 | ad 100 | ad 100 |
| additives | | | | | |
| | Formula | Formula | Formula | Formula | Formula |
| | 86 | 87 | 88 | 89 | 90 |
| Copolymer a) ³⁾ | 0.5 to 3.0 | 0.5 to 3.0 | 0.7 to 2.0 | 0.7 to 2.0 | 0.9 to 1.2 |
| Copolymer b) ⁴⁾ | 0.5 to 5.0 | 1.0 to 4.0 | 1.0 to 4.0 | 1.5 to 3.0 | 1.5 to 3.0 |
| Polyvinyl- | 1.0 to 10 | 2.0 to 9.0 | 2.5 to 8.5 | 2.0 to 8.0 | 3.0 to 7.0 |
| pyrrolidone | | | | | |
| and/or | | | | | |
| vinyl- | | | | | |
| pyrrolidone/ | | | | | |
| vinyl | | | | | |
| acetate | | | | | |
| copolymer | | | | | |
| Water | 80 to 99 | 82 to 99 | 84 to 98 | 87 to 98 | 90 to 97 |
| Optional | ad 100 | ad 100 | ad 100 | ad 100 | ad 100 |
| additives | | | | | |
| | Formula | Formula | Formula | Formula | Formula |
| | 91 | 92 | 93 | 94 | 95 |
| Acrylates/C₁₀₋₃₀ | 0.5 to 3.0 | 0.5 to 3.0 | 0.7 to 2.0 | 0.7 to 2.0 | 0.9 to 1.2 |
| Alkyl | | | | | |
| Methacrylate | | | | | |
| Copolymer | | | | | |
| Maltodextrin/ | 0.5 to 5.0 | 1.0 to 4.0 | 1.0 to 4.0 | 1.5 to 3.0 | 1.5 to 3.0 |
| VP Copolymer | | | | | |
| Polyvinyl- | 1.0 to 10 | 2.0 to 9.0 | 2.5 to 8.5 | 2.0 to 8.0 | 3.0 to 7.0 |
| pyrrolidone | | | | | |
| and/or | | | | | |
| vinyl- | | | | | |
| pyrrolidone/ | | | | | |
| vinyl | | | | | |
| acetate | | | | | |
| copolymer | | | | | |
| Optional | ad 100 | ad 100 | ad 100 | ad 100 | ad 100 |
| additives | | | | | |
| | Formula | Formula | Formula | Formula | Formula |
| | 96 | 97 | 98 | 99 | 100 |
| Acrylates/C₁₀₋₃₀ | 0.5 to 3.0 | 0.5 to 3.0 | 0.7 to 2.0 | 0.7 to 2.0 | 0.9 to 1.2 |
| Alkyl | | | | | |
| Methacrylate | | | | | |
| Copolymer | | | | | |
| Maltodextrin/ | 0.5 to 5.0 | 1.0 to 4.0 | 1.0 to 4.0 | 1.5 to 3.0 | 1.5 to 3.0 |
| VP Copolymer | | | | | |
| Polyvinyl- | 1.0 to 10 | 2.0 to 9.0 | 2.5 to 8.5 | 2.0 to 8.0 | 3.0 to 7.0 |
| pyrrolidone | | | | | |
| and/or | | | | | |
| vinyl- | | | | | |
| pyrrolidone/ | | | | | |
| vinyl | | | | | |
| acetate | | | | | |
| copolymer | | | | | |
| Water | 80 to 99 | 82 to 99 | 84 to 98 | 87 to 98 | 90 to 97 |
| Optional | ad 100 | ad 100 | ad 100 | ad 100 | ad 100 |
| additives | | | | | |

¹⁾ hydrophobically modified (meth)acrylic acid copolymer
²⁾ copolymer which is different from copolymer a) and which is obtained by reacting at least one ethylenically unsaturated monomer unit (b1) with at least one saccharide monomer unit (b2)
³⁾ copolymer a), which is obtained by reacting
a. at least one monomer (a1) selected from the group consisting of acrylic acid, methacrylic acid, C1-C6 alkyl acrylic acid esters, and C1-C6 alkyl methacrylic acid esters; with
b. at least one monomer (a2) selected from the group consisting of C10-30 alkyl acrylates, C10-30 alkyl methacrylates, C10-30 alkyl PEG acrylates, C10-30 alkyl PEG methacrylates and C10-30 alkyl PEG itaconates
⁴⁾ copolymer b), which is obtained by reacting N-vinylpyrrolidone (B1) with at least one monomer (B2) from the group of maltodextrins

As mentioned in the introduction, the cosmetic products described above are characterized by particular cosmetic properties on the hair, in particular advantageous properties for temporarily shaping the hair. A second subject matter of the present invention is therefore the use of a product according to the invention for temporarily shaping keratin-containing fibers, in particular human hair.

A third subject matter of the present invention is a method for temporarily shaping keratin-containing fibers, in particular human hair, in which a cosmetic product according to the invention is applied to the keratin fibers and the latter are temporarily fixed in shape.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

1. A cosmetic product for temporarily shaping keratin fibers, comprising, relative to the total weight of the product:
a) 0.5 to 3.0 wt. % of at least one hydrophobically modified (meth)acrylic acid copolymer;
b) 0.5 to 5.0 wt. % of at least one copolymer which is different from copolymer a) and which is obtained by reacting at least one ethylenically unsaturated monomer unit (b 1) with at least one saccharide monomer unit (b2).

2. The product according to claim 1, wherein the proportion by weight of the copolymer a) in the total weight of the cosmetic product is 0.7 to 2.0 wt %.

3. The product according to claim 1, wherein the proportion by weight of the copolymer a) in the total weight of the cosmetic product is 0.9 to 1.2 wt %.

4. The product according to claim 1, wherein the copolymer a) is obtained by reacting
at least one monomer (a1) selected from the group consisting of acrylic acid, methacrylic acid, C1-C6 alkyl acrylic acid esters, and C1-C6 alkyl methacrylic acid esters; with
at least one monomer (a2) selected from the group consisting of C10-30 alkyl acrylates, C10-30 alkyl methacrylates, C10-30 alkyl PEG acrylates, C10-30 alkyl PEG methacrylates, and C10-30 alkyl PEG itaconates.

5. The product according to claim 1, wherein the copolymer a) is selected from the group consisting of: compounds having the INCI name Acrylates/C10-30 Alkyl Methacrylate Copolymer.

6. The product according to claim 1, wherein the proportion by weight of the copolymer b) in the total weight of the cosmetic product is 1.0 to 4.0 wt %.

7. The product according to claim 1, wherein the proportion by weight of the copolymer b) in the total weight of the cosmetic product is 1.5 to 3.0 wt %.

8. The product according to claim 1, wherein the copolymer b) is obtained by reacting
at least one monomer (b 1) from the group consisting of N-vinylpyrrolidone and N-vinylformamide, with
at least one monomer (b2) from the group consisting of modified starches.

9. The product according to claim 1, wherein the copolymer b) is a compound with the INCI name Maltodextrin/VP Copolymer.

10. The product according to claim 1, further comprising relative to the total weight of the product, c) 1.0 to 10 wt.-% polyvinylpyrrolidone and/or vinylpyrrolidone/vinyl acetate copolymer.

11. The product according to claim 10, wherein the proportion by weight of the polyvinylpyrrolidone and/or vinylpyrrolidone/vinyl acetate copolymer c) in the total weight of the cosmetic product is 2.0 to 8.5 wt %.

12. The product according to claim 10, wherein the proportion by weight of the polyvinylpyrrolidone and/or vinylpyrrolidone/vinyl acetate copolymer c) in the total weight of the cosmetic product is 3.0 to 7.0 wt %.

13. The product according to claim 1, further comprising by total weight of the product c) 1.0 to 10 wt % polyvinylpyrrolidone.

14. The product according to claim 1, comprising, relative to the total weight of the product:
a) 0.5 to 3.0 wt.-% of a copolymer a), which is obtained by reacting
at least one monomer (a1) selected from the group consisting of acrylic acid, methacrylic acid, C1-C6 alkyl acrylic acid esters, and C1-C6 alkyl methacrylic acid esters; with
at least one monomer (a2) selected from the group consisting of C10-30 alkyl acrylates, C10-30 alkyl methacrylates, C10-30 alkyl PEG acrylates, C10-30 alkyl PEG methacrylates, and C10-30 alkyl PEG itaconates;
b) 0.5 to 5.0 wt.-% of a copolymer b), which is obtained by reacting N-vinylpyrrolidone (b1) with at least one maltodextrin monomer (b2);
c) 1.0 to 10 wt.-% polyvinylpyrrolidone.

15. The product according to claim 1, comprising, relative to the total weight of the product:
d) 0.9 to 1.2 wt % of a copolymer a) from the group of compounds having the INCI name Acrylates/C10 30 Alkyl Methacrylate Copolymer;
e) 1.5 to 3.0 wt % of a copolymer b) from the group of compounds having the INCI name Maltodextrin/VP Copolymer;
f) 3.0 to 7.0 wt-% polyvinylpyrrolidone.

16. The product according to claim 1, wherein the product further comprises, relative to the total weight of the product, 80 to 99 wt % water.

17. The product according to claim 1, wherein the product further comprises, relative to the total weight of the product 84 to 98 wt % water.

18. The product according to claim 1, wherein the product further comprises, relative to the total weight of the product 90 to 97 wt % water.

19. A method for temporarily shaping keratin-containing fibers, applying to keratin fibers the cosmetic product according to claim 1.
